(19) United States
(12) Patent Application Publication
(10) Pub. No.: US 2014/0276235 A1
(43) Pub. Date: Sep. 18, 2014

(54) BIOFEEDBACK SYSTEMS AND METHODS

(71) Applicant: FIRST PRINCIPLES, INC., ALBANY, NY (US)

(72) Inventor: Keith A. Raniere, Clifton Park, NY (US)

(73) Assignee: FIRST PRINCIPLES, INC., ALBANY, NY (US)

(21) Appl. No.: 13/841,309

(22) Filed: Mar. 15, 2013

(51) Int. Cl. A61B 5/00; A61B 5/103

(52) U.S. Cl. CPC A61B 5/6829; A61B 5/486; A61B 5/1038
USPC 600/592

(57) ABSTRACT

Systems and methods for constructing custom orthotics are described. Several embodiments of the system use sensor pads to obtain both static and dynamic three dimensional information concerning the shape or topography of the surface of a patient's foot and/or ankle. The information is analyzed to obtain information useful in constructing a custom orthotic from a selection of basic orthotic shells. Once constructed, the orthotic may modify a patient's gait. One embodiment of the present disclosure includes a user terminal including a plurality of sensors in a fitted material, the sensors being connected to a computer, a computer configured to analyze three dimensional information acquired by the plurality of sensors, a computer and/or a manufacturing terminal configured to display the results of the computer's analysis of the three dimensional information and a network that connects the user terminal to the computer and the computer to the manufacturing terminal.

### BACKGROUND OF THE DISCLOSURE

The present disclosure relates generally to the field of biofeedback including information capture and more particularly to the capture of the three dimensional shape of a human body part, such as, for example, a foot and, even more particularly, to the use of captured three dimensional shape information to produce orthotics.

Orthotics are shoe inserts that are intended to correct an abnormal or irregular walking pattern. Orthotics perform functions that make standing, walking and running more comfortable and efficient by altering slightly the angles at which the foot strikes a surface. Orthotics take various forms and are constructed from various materials. Orthotics are generally concerned with improving foot function and minimizing stress forces that could ultimately cause foot deformity and pain.

Since they are the major weight-bearing part of the body, foot pain is common; half of the Americans polled by the American Podiatric Medical Association had missed a day of work because of foot problems. The foot contains 26 bones, up to two sesamoid bones and many small structures which support and balance the weight of the entire body. Walking puts up to 1.5 times one's body weight on the foot and humans walk an average of 1000 miles per year. Foot pain is not normal and should not be ignored; problems can affect the functioning of other parts of the body, including the hips, knees and back. Various practitioners will often recommend custom foot orthoses as part of a treatment regime in order to: reduce the symptoms associated with many foot related pathologies; provide support; accommodate foot deformity; provide better positioning; relieve pressure on a certain area of the foot; and improve the overall biomechanical function of the foot and lower extremity.

A rigid orthotic is an orthotic designed to control foot function and can be made of a firm material such as plastic or carbon fiber. Rigid orthotics are often designed to control motion in two major foot joints, which lie directly below the ankle joint. This type of orthotic is commonly recommended by physicians in response to strains, aches and pains in the legs, thighs, and lower back. Rigid orthotics are generally fabricated from a plaster of paris mold of an individual foot. The finished orthotic normally extends along the sole of the heel to the ball or toes of the foot.

Soft orthotics can be used to absorb shock, increase balance and relieve pressure from sore spots. Soft orthotics are typically constructed from soft, compressible materials and may be molded by the action of the foot in walking or fashioned over a plaster impression of the foot. A useful aspect of soft orthotics is that they may be easily adjusted to changing weight-bearing forces. However, material wear can require that they be frequently replaced. Use of soft orthotics has been shown to be effective for treating arthritis sufferers, people with foot deformities and patients suffering from diabetic foot. Soft orthotics are typically worn against the sole of the foot and extend from the heel past the ball of the foot to include the toes.

Semirigid orthotics provide for dynamic balance of the foot while walking or participating in sports. When used for participating in sports, the nature of the sport can impact upon the orthotic design. The purpose of a semirigid orthotic is to help guide the foot through proper functions, allowing the muscles and tendons to perform more efficiently. A basic semirigid orthotic can be constructed from layers of soft material that are reinforced with more rigid materials.

Orthotics have typically been constructed by using casting materials to take a mold of the subject's foot. The mold is then used to construct an orthotic that conforms to the base of the subject's foot. Various other orthotics may be used for multidirectional sports or edge-control sports by casting the foot within the shoe, such as a ski boot, ice skate boot, or inline skate boot.

### SUMMARY OF THE DISCLOSURE

Embodiments of the present disclosure may include a biofeedback apparatus including a fitted material operatively wearable by a user, a plurality of sensing units operably associated with the fitted material, at least one sensing unit transceiver for each sensing unit of the plurality of sensing units, wherein the at least one sensing unit transceiver is operatively associated with said fitted material and said plurality of sensing units, a computer having a transceiver for communicating with the biofeedback apparatus; and a computing capability of the computer for analyzing the object data. The biofeedback apparatus may be configured to acquire three dimensional information concerning the shape of a user's foot, both statically and dynamically. The three dimensional information may be provided to the computer that analyzes the information and the analyzed information may be provided to a manufacturing terminal, where a technician may use the information to select and shape an orthotic shell. Alternatively, the information may enable the automated manufacture of a custom orthotic. In one aspect of the present disclosure, custom orthotics may be constructed that modify the gait of a patient.

One specific embodiment of the present disclosure includes a biofeedback system and method having a biofeedback apparatus including a fitted material wearable on a user's foot or other body part, the fitted material including a plurality of sensing units operatively associated with the fitted material, at least one sensing unit transceiver for each sensing unit of the plurality of sensing units wherein the at least one sensing unit transceiver being operatively associated with the fitted material and the plurality of sensing units, a data acquisition device for acquiring at least one object data from the plurality of sensors, a computer configured to communicate with the biofeedback apparatus and being capable of analyzing the at least one object of data, such as, for example, three dimensional information acquired by the plurality of sensing units, a manufacturing terminal configured to display the results of the computers analysis of the at least one object of data, such as, the three dimensional information and a network that connects the biofeedback apparatus to the computer.

In a specific embodiment, a systems and methods for wearing a sock with sensors to detect and record changes in pressure and or tension in a foot/feet or other body part both statically and dynamically may be utilized.

In another embodiment, the fitted material further comprises a woven material

In a further embodiment, at least a portion of each sensing units is removably attached to the fitted material.

In yet another embodiment, the data acquisition device comprises a media storage device.

In a still further embodiment, the data acquisition device further comprises a wireless connection to an input device of the computer.

In another embodiment, the data acquisition device further comprises a wire connection between an input device of the computer and the biofeedback apparatus.

In a further embodiment, the computing capability further comprises a computer program.

In yet another embodiment, the sensing unit further comprises one or more of the following, selected from the group consisting of: a pressure sensor, strain sensor, flex sensor, displacement sensor, and combinations thereof.

In a still further embodiment, data acquisition device comprises a wireless communicator to a remote location.

In another embodiment, the sensing unit transceiver of a first sensing unit communicates to the sensing unit transceiver of a second sensing unit of the plurality of sensing units.

In a further embodiment, the fitted material further is selected from a group consisting of: a textile, a plastic, a polymer, a felt, a spunlace, a gauze, a nylon, a vinyl, a metal, and a combination thereof.

In yet another embodiment, the fitted material is a sleeve, with at least one open end.

In a still further embodiment, a biofeedback method comprises: providing a biofeedback apparatus, the biofeedback apparatus having: a fitted material configured to a user's body part, such as, for example, a foot; a plurality of sensing units operably associated with said fitted material; a plurality of sensing unit transceivers operatively associated to each sensing unit of the plurality of sensing units, wherein said at least one sensing unit transceiver is operatively associated with said fitted material and said plurality of sensing units; and a data acquisition device, for acquiring at least one object data from said plurality of sensing unit transceivers; fitting said biofeedback apparatus onto the user's body part, such as, for example, a foot; locomoting said biofeedback apparatus with said user's body part, such as, for example, a foot; sensing at least one variable with the plurality of sensing units; communicating the at least one variable to a computer configured with computation programming; and analyzing the object data by the computer to determine a biofeedback for the body part, such as, for example, a foot.

In another embodiment, the method comprises preparing an incorporated biofeedback orthotic which incorporates the biofeedback.

In a further embodiment, the method comprises sensing at least a plurality of locations with the biofeedback apparatus

In yet another embodiment, the method comprises at least one variable which may be selected from the group consisting essentially of: pressure, displacement, strain, stress, static friction, kinetic friction, and combinations thereof

In another embodiment, the computer may be configured to determine a center of balance from the three dimensional information.

In a further embodiment, the computer may be configured to determine a gait line from the three dimensional information.

In yet another embodiment, the computer may be configured to determine an arch height from the three dimensional information.

In a still further embodiment, the three dimensional information may include a single array of data describing the topography of a patient's body part, such as, for example, a foot.

In another embodiment again, the three dimensional information may include a plurality of arrays of data describing the topography of the portions of a patient's body part, such as, for example, a foot contacting the sensors operatively associated with the fitted material during dynamic motion.

In a still further embodiment again, the computer may utilize the plurality of arrays to identify the time spent in the contact, midstance and propulsive phases of a gait cycle.

An embodiment of the systems and methods of the present disclosure may include acquiring three dimensional information concerning the shape of a patient's body part, such as, for example, in a foot, analyzing the three dimensional information and displaying the three dimensional information and the analysis.

In a further embodiment of the systems and methods of the present disclosure, the three dimensional information may include information acquired while the patient is stationary and information acquired while the patient is walking.

In another embodiment of the systems and methods of the present disclosure, the analysis may include information concerning the patient's center of balance.

In a still further embodiment of the systems and methods of the present disclosure, the analysis may include information concerning the patient's gait line.

In yet another embodiment of the systems and methods of the present disclosure, the analysis may include information concerning the patient's arch.

In a still further embodiment of the systems and methods of the present disclosure, the analysis may include the proportion of time spent in the contact, midstance and propulsive phases of a gate cycle.

In yet another embodiment of the systems and methods of the present disclosure, the display may be in the form of a printed information sheet.

In a still further additional embodiment, the display may be in the form of a graphical display on a computer screen.

In a yet another additional embodiment, the three dimensional information may be displayed in a plurality of different ways.

### BRIEF DESCRIPTION OF THE DRAWINGS

FIG. 1 is a schematic diagram of a Biofeedback System useful with an embodiment of the present disclosure;

FIG. 2 is a schematic diagram of a Biofeedback apparatus useful with an embodiment of the present disclosure;

FIG. 3 is a schematic diagram of a sock, part of the Biofeedback apparatus of FIG. 2 useful with an embodiment of the present disclosure;

FIG. 4 is a flow diagram illustrating a method of capturing three dimensional information concerning the shape of a foot useful with an embodiment of the method of the present disclosure;

FIG. 5 a flow diagram illustrating a method of dynamically capturing three dimensional information concerning the shape of a foot in motion useful with an embodiment of the method of the present disclosure;

FIG. 6 is a schematic view of a three dimensional contour map generated useful with an embodiment of the present disclosure;

FIG. 7 is a flow diagram illustrating a process for generating a three dimensional contour map useful with an embodiment of the present disclosure

FIG. 8 is a flow diagram illustrating a process for generating a two dimensional interpolated height information display useful with an embodiment of the method of the present disclosure;

FIG. 9 is a flow diagram illustrating a process for calculating the location of the center of mass of the force exerted on a footpad by the patient's foot;

FIG. 10 is a flow diagram illustrating a process for displaying information concerning the shape of a foot during dynamic contact with a sensing surface useful with an embodiment of the method of the present disclosure;

FIG. 11 is a flow diagram illustrating a process for generating a “gait line” useful with an embodiment of the method of the present disclosure;

FIG. 12 is a flow diagram illustrating a process for storing patient information and transferring the information to a server useful with an embodiment of the method of the present disclosure; and

FIG. 13 is a flow diagram illustrating a process useful with the present disclosure for receiving and storing patient information transmitted by a user terminal over a network useful with an embodiment of the method of the present disclosure.

### DESCRIPTION OF THE DISCLOSURE

Turning now to the drawings, biofeedback systems and methods for obtaining information that may be useful in the manufacture of orthotics is illustrated. One aspect of the present disclosure may involve a biofeedback system for collecting data, such as, for example, three dimensional information using a plurality of sensors concerning the shape of a patient's foot or other body part, both statically and dynamically, analyzing the data and transmitting the information to a manufacturing facility or other appropriate location, although the disclosure is not limited in this regard. The patient information may be collected using a plurality of sensors operatively positioned in a fitted material wearable on a user's foot, then processed by a computer for printing/and or display and possibly being transmitted over telephone lines or the internet, although the disclosure is not limited in this regard. A manufacturing facility may receive the transmitted information and the information used to generate an orthotic, although the disclosure is not limited in this regard.

Systems and methods useful with one embodiment of the present disclosure include collecting three dimensional information concerning the shape of a patient's body part, such as, for example, a foot and using the information to manufacture orthotics is illustrated in FIG. 1. The biofeedback system 30 may include at least one biofeedback apparatus 32 including a computer/server 31 and may include at least one wearable fitted material 39 operatively positioned on a patient's body such as, for example, a sock 34 having a plurality of sensors 36 operatively positioned within the fitted material 39 which comprises the sock 34 or otherwise operatively coupled to the fitted wearable material 39 comprising the sock 34, to detect and record changes in pressure and or tension in a foot/feet both statically and dynamically as required, although the scope of the disclosure is not limited in this regard. The biofeedback apparatus 32 may be connected to a network 37, although the scope of the disclosure is not limited in this regard. A manufacturing terminal 40 may also connected to the network 37, although the scope of the disclosure is not limited in this regard.

The biofeedback apparatus 32 may collect information about a patient's body part, such as, for example, a foot using the sock 34 having the plurality of sensors 36 operatively associated therewith to detect and record changes in pressure and or tension in the body part, such as, for example, a foot, although the scope of the disclosure is not limited in this regard. The information may be processed by the biofeedback apparatus 32 and then sent to a computer/server 38 via the network 37, although the scope of the disclosure is not limited in this regard. The computer/server 38 may receive process and store the information and then may provide the information to the manufacturing terminal 40, although the scope of the disclosure is not limited in that regard. A lab technician may use the manufacturing terminal to determine the appropriate construction of an orthotic, although the scope of the disclosure is not limited in this regard.

A biofeedback apparatus 32 useful with an embodiment of the present disclosure is illustrated in FIG. 2, although the scope of the disclosure is not limited in this regard. The biofeedback apparatus 32 may include a computer 31. The computer 31 may be connected to a sock 34 having sensors 36 to detect and record changes in pressure and or tension in a body parts such as, for example, a foot/feet and a modem 42, although the scope of the disclosure is not limited in this regard. The modem 42 may be connected to a telephone line 44, although the scope of the disclosure is not limited in this regard. In one embodiment, the biofeedback system 30 may be a self activated kiosk in a retail outlet, although the scope of the disclosure is not limited in this regard. In another embodiment, the biofeedback system 30 apparatus may be a station located in a doctor's office, although the scope of the disclosure is not limited in this regard. Stations located in doctor's offices may contain some of the functionality attributed to other components of the system useful with the present disclosure such as the server 38 and/or the manufacturing terminal 40, although the scope of the disclosure is not limited in this regard.

In several embodiments, the biofeedback apparatus 32 may capture three dimensional information concerning the shape of the patient's foot using the sock 34 having sensors 36 to detect and record changes in pressure and or tension in a body part, such as, for example, a foot/feet, although the scope of the disclosure is not limited in this regard. The captured information may then be displayed on the biofeedback apparatus 32 or transferred to another computer over a telephone line using 44 the modem 42, although the scope of the disclosure is not limited in this regard. In other embodiments, the biofeedback apparatus 32 may be connected to a network 37 via the internet, a network interface card, cable modem or similar network interface device, although the scope of the disclosure is not limited in this regard.

At least one sock 34 having sensors 36 to detect and record changes in pressure and or tension in a body part, such as, for example, a foot/feet useful with an embodiment of the present disclosure is illustrated in FIG. 3, although the scope of the disclosure is not limited in this regard. The sock 34 may include sensors 36 to detect and record changes in pressure and or tension in a foot. An array of sensors 36 are located throughout the sock 34 wherever the sock 34 contacts the foot or body of the patient, including but not limited to, the sole of the foot and the ankle, although the scope of the disclosure is not limited in this regard. The array of sensors 36 may be connected to an analog-to-digital converter or other conventional electronic device, although the scope of the disclosure is not limited in this regard. The analog-to-digital converter may have an output, although the scope of the disclosure is not limited in this regard.

In one embodiment, the sensors 36 may be fabric-based sensors such as those disclosed in U.S. Pat. No. 6,970,731 to Jayaraman et al., the disclosure of which is hereby incorporated by reference to the extent not inconsistent with the present disclosure, although the scope of the disclosure is not limited in this respect. The material having the sensors 36 must fit snugly (sock or sleeve) to the body part that is being measured. An example of alternative type electronic resistor user interface flexible conductive materials that may be used with the present disclosure includes, but is not limited to, the flexible switching devices disclosed in U.S. Pat. No. 7,145,432 to Lussey et al., the disclosure of which is hereby incorporated by reference to the extent not inconsistent with the present disclosure, although the scope of the disclosure is not limited in this respect. The apparatus and method of pressure and tension fitting around/about one's foot in order to fit for orthotics will now be described.

In a specific system and method, patients may place one or both of their feet in the sock or socks 34. A plurality of sensors 36 may be positioned in the material around/about a foot/joint, although the scope of the disclosure is not limited in this respect. Communications may be maintained between the plurality of sensors 36 and the biofeedback apparatus computer 31 for monitoring impulses from and sending impulses to the plurality of sensors 36. The sock/sleeve device 34 may be expandable and contractible to provide continuous contact with more areas of the foot/body. The object is to map the pressure/tension caused about/around the body part, such as, for example, the foot while the body part is in motion as a result of the interplay of muscles, bones, tendons, ligaments, etc. and orientation, although the scope of the disclosure is not limited in this respect.

Orthotic manufacturing equipment may be located at a manufacturing facility, although the scope of the disclosure is not limited in this respect. One embodiment that may be useful with the present disclosure is illustrated in FIG. 4 of U.S. Pat. No. 7,346,419 to Lowe, the disclosure of which is hereby incorporated by reference to the extent not inconsistent with the present disclosure, although the scope of the disclosure is not limited in this respect.

The computer 31 may receive the communications from the sensors 36 and convert the communications into three dimensional information concerning the shape of a patient's body part, such as, for example, a foot and performs analysis of this information to generate parameters that may be useful in the manufacturing process of an orthotic, although the scope of the disclosure is not limited in this respect. The information and the parameters may then be transferred via the network 37 to the manufacturing terminal 40, where a technician may view the information and be guided by the generated parameters in the selection of an orthotic shell that may then be modified to create an orthotic customized to the shape of the patient's foot, as would be understood by those skilled in the art, although the scope of the disclosure is not limited in this respect.

In one embodiment of the systems and methods of the present disclosure, the technician may have a number of different types of orthotic shells that may be used to create custom orthotics and the most appropriate shell may be indicated by the parameters determined by the computer from the three dimensional information of the patient's foot, although the scope of the disclosure is not limited in this respect.

Orthotic shells useful with an embodiment of the present disclosure are well-known in the art and the need to illustrate same is believed unnecessary. The shell may be modified using a heat gun or similar device to increase or decrease arch height or modify any other aspect of the orthotic shell's shape, as would be understood by those skilled in the art, although the scope of the disclosure is not limited in this respect.

The hardware described above may be operated in conjunction with software, although the scope of the disclosure is not limited in this respect. The following may provide a description of possible various software routines that may be used useful with embodiments of the present disclosure to operate the hardware described above, although the scope of the disclosure is not limited in this respect.

The biofeedback apparatus 32 may capture three dimensional information concerning the shape of a patient's foot. In one embodiment, software may enable the hardware to capture this information both statically and dynamically. A flow chart illustrating a process that may be implemented using the hardware described above and software for capturing three dimensional information of the shape of a patient's foot useful with an embodiment of the present disclosure is illustrated in FIG. 4. The process 70 may include detecting at 72 that a patient has a sock having the plurality of sensors on the appropriate body part/foot, although the scope of the disclosure is not limited in this respect. In one embodiment, an analog-to-digital converter may be normalized at 74 using the bisection method to search the range of the analog-to-digital converter for the highest sensitivity level at which the analog-to-digital converter is not saturated, although the scope of the disclosure is not limited in this respect. Once the analog-to-digital converter has been normalized, a sample of all the sensors 36 may be taken at 76, although the scope of the disclosure is not limited in this respect. The samples are stored at 78 as an array in memory, although the scope of the disclosure is not limited in this respect. The process is then repeated with increasing at 82 levels for the analog-to-digital converter until the maximum level of the analog-to-digital converter is reached at 80, although the scope of the disclosure is not limited in this respect. Once the maximum level has been reached, the measurement may be completed at 84, although the scope of the disclosure is not limited in this respect.

The normalization of the analog-to-digital converter enables the system to choose the level of sensitivity that provides the greatest amount of information for each patient, although the scope of the disclosure is not limited in this respect. A heavier person will saturate many of the sensors 36 at a high level of analog-to-digital converter sensitivity and a lighter person will generate currents that appear uniform at a low level of analog-to-digital converter sensitivity, although the scope of the disclosure is not limited in this respect. By using the bisection method to locate the maximum sensitivity of the analog-to-digital converter, a data set may be obtained that possesses a significant range of values without saturation, although the scope of the disclosure is not limited in this respect.

A flow chart illustrating a process that enables the capture of three dimensional information concerning the shape of a patient's foot, when the foot is in motion, is shown in FIG. 5. The process 100 may include normalizing the analog-to-digital converter in the manner described above and then scanning at 102 the sensors 36, although the scope of the disclosure is not limited in this respect. If no pressure/tension is detected, then the process pauses at 103 and a new scan is taken until pressure/tension is detected, although the scope of the disclosure is not limited in this respect. Once pressure/tension may be detected at 104, the scanned data may be stored at 106, a timer may be started and the process pauses at 107 before scanning at 108 the sensors 36 again, although the scope of the disclosure is not limited in this respect. The scan may be stored if pressure is detected at 110, although the scope of the disclosure is not limited in this respect. The process continues to scan and store data until sensor pressure/tension is no longer detected at 110 or the timer times out, although the scope of the disclosure is not limited in this respect.

In addition to capturing information using the sock sensor 36, the computer 31 useful with an embodiment of the present disclosure may display the captured information, although the scope of the disclosure is not limited in this respect. The information may be displayed in one of a number of manners. In one embodiment, three dimensional information concerning the shape of a patient's foot may be displayed as a two dimensional height information display, a two dimensional interpolated height information display or a three dimensional map, although the scope of the disclosure is not limited in this respect.

A representative example of a three dimensional map that may be generated useful with an embodiment of the present disclosure is illustrated in FIG. 6. The image 190 may include a three dimensional contour map of each foot, although the scope of the disclosure is not limited in this respect. The contour map may use a combination of contour lines and color to create the illusion of a three dimensional surface on the two dimensional computer screen, although the scope of the disclosure is not limited in this respect. The contour lines and the colors may be chosen to represent a three dimensional shape corresponding to the surface of the patient's foot, although the scope of the disclosure is not limited in this respect.

A process for generating a three dimensional contour map useful with an embodiment of the present disclosure is illustrated in FIG. 7. The process 200, useful with the present disclosure, may include retrieving at 202 the scan information and generating at 204 a contour map using adjacent data points, although the scope of the disclosure is not limited in this respect. In one embodiment, a commercial contour mapping engine such as, for example, TeeChart may be used to generate the contour map, although the scope of the disclosure is not limited in this respect. The retrieved information may also be used to generate at 206 an interpolated data set in the manner described in FIG. 6. Once colors are assigned to the interpolated data set to represent the relative height of each of the data points, the interpolated data set may be superimposed onto the contour map, although the scope of the disclosure is not limited in this respect. The contour map and the superimposed interpolated data set may then the displayed at 210, although the scope of the disclosure is not limited in this respect.

Referring back to FIG. 6, the center of balance 196 may be shown as black dot superimposed on the image of the patient's foot, although the scope of the disclosure is not limited in this respect. A process that may be used useful with one embodiment of the present disclosure to calculate the location of the center of mass of the force exerted on the sock by the patient's foot is illustrated in FIG. 9, although the scope of the disclosure is not limited in this respect. The process 220 may involve taking at 222 a weighted average of the grid co-ordinates of the data points, although the scope of the disclosure is not limited in this respect. Each grid location may be weighted according to the amount of pressure exerted on that grid cell by the patient's foot, although the scope of the disclosure is not limited in this respect. The amount of pressure is determined using the height data collected using the sock, although the scope of the disclosure is not limited in this respect. The weighted average may be the center of balance, although the scope of the disclosure is not limited in this respect. The grid location of the center of balance may be determined at 224 and then superimposed at 226 over the height information display, although the scope of the disclosure is not limited in this respect. The center of balance may be superimposed over whichever display mode may be used to display the height information collected by the sock, although the scope of the disclosure is not limited in this respect.

As discussed above, a biofeedback apparatus 32 useful with an embodiment of the present disclosure is capable of capturing information dynamically, although the scope of the disclosure is not limited in this respect. Dynamic information capture may be used to obtain information concerning the manner in which the undersurface of a patient's foot changes shape as the patient walks or runs, although the scope of the disclosure is not limited in this respect.

An embodiment of a process for obtaining information concerning the shape of a patient's foot during motion is illustrated in FIG. 5, although the scope of the disclosure is not limited in this respect. A process useful with an embodiment of the present disclosure for displaying information concerning the shape of a patient's foot during motion is illustrated in FIG. 10, although the scope of the disclosure is not limited in this respect. The process 240 includes retrieving at 242 a first frame of stored data, although the scope of the disclosure is not limited in this respect. The frame of data may be then processed to generate an image in a desired format 244, although the scope of the disclosure is not limited in this respect. Typically the desired format may be either a two dimensional interpolated height information display or a three dimensional contour map, although the scope of the disclosure is not limited in this respect. The two dimensional interpolated height information display or the three dimensional contour map may be generated useful with the description provided above, although the scope of the disclosure is not limited in this respect. The image frame is then stored at 246 and a determination at 248 may be made as to whether any additional frames of data were captured, although the scope of the disclosure is not limited in this respect. If additional frames of data exist, then each of these frames is retrieved and an image in the desired format is generated and stored, although the scope of the disclosure is not limited in this respect. The process repeats until no additional frames of data remain, although the scope of the disclosure is not limited in this respect. Once images have been generated from each of the frames of data, then the sequence of image frames may be displayed at 250 on a computer screen, although the scope of the disclosure is not limited in this respect. With sufficient processing power, the display of the image frames may occur simultaneously with the generation of the images for later frames of data, although the scope of the disclosure is not limited in this respect.

In addition to displaying information concerning the shape of a patient's foot, biofeedback system 30 is useful with the present disclosure may be capable of analyzing a patient's gait, although the scope of the disclosure is not limited in this respect. During the display of the dynamic information, the biofeedback system 30 may show the location of the center of balance in each frame, display the elapsed time, the percentage and duration of time spent on three important phases of the gait cycle (i.e. the contact, midstance and propulsive phases) and/or the “gait line”, which may be a composite of all of the center of balance for each frame of the dynamic information, although the scope of the disclosure is not limited in this respect.

In one embodiment, the center of balance in each frame is calculated using the process is illustrated in FIG. 9, although the scope of the disclosure is not limited in this respect. The “gait line” is simply a line corresponding to the change in the location of the center of balance as the patient's foot contacts wearing the sock contacts a solid surface and then lifts from the solid surface, although the scope of the disclosure is not limited in this respect. An embodiment of a process useful with the present disclosure for generating a “gait line” from frame data captured using a sock may be illustrated in FIG. 11, although the scope of the disclosure is not limited in this respect. The process 260 may include retrieving at 262 a first frame of data, although the scope of the disclosure is not limited in this respect. The retrieved data may be used to generate at 264 an image frame in a desired format in a similar manner to that discussed above, although the scope of the disclosure is not limited in this respect. The center of balance for the frame may then calculated using the retrieved data in a manner similar to that described above in relation to FIG. 9, although the scope of the disclosure is not limited in this respect. The location of the center of balance may then used to form the “gait line”, although the scope of the disclosure is not limited in this respect. The “gait line” may start at the location of the center of balance for the initial frame and then may be formed at 268 by extrapolating from the center of mass from the previous frame to the center of mass of the current frame, although the scope of the disclosure is not limited in this respect. The “gait line” may be then superimposed at 270 on the image frame and the result may be stored at 272, although the scope of the disclosure is not limited in this respect. If there are additional frames of data at 274, then the process may be repeated, although the scope of the disclosure is not limited in this respect. Otherwise the sequence of image frames may be sequentially displayed at 276 on a computer screen, although the scope of the disclosure is not limited in this respect.

In other embodiments, a similar process may be used simply to generate the “gait line” without generating the image frame information, although the scope of the disclosure is not limited in this respect. The “gait line” may then be superimposed on a static image of the patient's foot, although the scope of the disclosure is not limited in this respect.

As described above, the biofeedback apparatus 32 may store the raw information obtained from the sock 34 in a database and may then transmit the information to a server 38, although the scope of the disclosure is not limited in this respect. An embodiment of a process useful with the present disclosure for storing the information and then transferring the information to a server is illustrated in FIG. 12, although the scope of the disclosure is not limited in this respect. The process 320 may include retrieving at 322 the stored information that may be sent to the server, although the scope of the disclosure is not limited in this respect. The retrieved information may be compressed at 324, a connection may be established with a server 326 and the compressed information may be transferred at 328 to the server using a file transfer protocol, as would be understood by those skilled in the art, although the scope of the disclosure is not limited in this respect.

In other embodiments, other techniques involving the transfer of digital information may be used to transfer the three dimensional information concerning the shape of a patient's foot to a server, although the scope of the disclosure is not limited in this respect. In other embodiments, additional information such as the image information that may be displayed using a computer is also transferred, although the scope of the disclosure is not limited in this respect.

As discussed above, a server 38 may receive information transmitted by the biofeedback apparatus 32, stores the information in a database, performs operations to obtain custom fitting parameters and transfers the information and the custom fitting parameters to a manufacturing terminal 40, although the scope of the disclosure is not limited in this respect.

An embodiment of a process useful with the present disclosure for receiving and storing information transmitted by a biofeedback system 30 over a network 37 is illustrated in FIG. 13, although the scope of the disclosure is not limited in this respect. The process 340 may include receiving at 342 a request to initiate a file transfer. Receiving at 344 a file transferred that may use a file transfer protocol, although the scope of the disclosure is not limited in this respect. Decompressing at 346 the file to yield three dimensional information concerning a patient's foot and then storing at 348 the information in a database, although the scope of the disclosure is not limited in this respect.

Methods similar to those illustrated in FIGS. 12 and 13 may be used to transfer data between a server and a manufacturing terminal, although the scope of the disclosure is not limited in this respect.

A computer 31 useful with an embodiment of the present disclosure may analyze the three dimensional information concerning the shape of a patient's foot that may be provided by a biofeedback apparatus 32, although the scope of the disclosure is not limited in this respect. In one embodiment, the computer 31 analyzes the three dimensional information to obtain custom fitting parameters such as the arch height of the patient's foot, center of balance for each foot and center of balance for the patient, although the scope of the disclosure is not limited in this respect. In several embodiments, batch processing of three dimensional information may be performed, although the scope of the disclosure is not limited in this respect. In other embodiments, three dimensional information may be analyzed as it may be received, although the scope of the disclosure is not limited in this respect.

Although the foregoing embodiments are disclosed, it would be understood that additional variations, substitutions and modifications may be made to the system, as disclosed, without departing from the scope of the present disclosure. Accordingly, the scope of the present disclosure should be determined not by the embodiments illustrated, but by the appended claims.

I claim:

1. A biofeedback system, comprising:
a biofeedback apparatus, having:
a fitted material operatively wearable by a user;
a plurality of sensing units operably associated with the fitted material;
at least one sensing unit transceiver for each sensing unit of the plurality of sensing units, wherein the at least one sensing unit transceiver is operatively associated with said fitted material and the plurality of sensing units;
a data acquisition device, for acquiring at least one object data from the plurality of sensors;
a computer having a transceiver for communicating with the biofeedback apparatus; and
a computing capability of the computer for analyzing the object data.

2. The biofeedback system of claim 1, wherein the fitted material further comprises a woven material.

3. The biofeedback system of claim 1, wherein at least a portion of each sensing units is removably attached to the fitted material.

4. The biofeedback system of claim 1, wherein the data acquisition device comprises a media storage device.

5. The biofeedback system of claim 1, wherein the data acquisition device further comprises wireless connection to an input device of the computer.

6. The biofeedback system of claim 1, wherein the data acquisition device further comprises a wire connection between an input device of the computer and the biofeedback apparatus.

7. The biofeedback system of claim 1, wherein the computing capability further comprises a computer program.

8. A biofeedback apparatus comprising:
a fitted material wearable on a user's foot;
a plurality of sensing units operably associated with said fitted material;
at least one sensing unit transceiver for each sensing unit of the plurality of sensing units, wherein said at least one sensing unit transceiver is operatively associated with said fitted material and said plurality of sensing units; and
a data acquisition device, operatively associated with the at least one sensing unit transceiver, for acquiring at least one object data from said plurality of sensors.

9. The apparatus of claim 8, wherein the sensing unit further comprises one or more of the following, selected from the group consisting of: a pressure sensor, strain sensor, flex sensor, displacement sensor, and combinations thereof.

10. The apparatus of claim 8, wherein the data acquisition device comprises a media storage device.

11. The biofeedback apparatus of claim 8, wherein the data acquisition device comprises a wireless communicator to a remote location.

12. The biofeedback apparatus of claim 8, wherein the sensing unit transceiver of a first sensing unit communicates to the sensing unit transceiver of a second sensing unit of the plurality of sensing units.

13. The biofeedback apparatus of claim 8, wherein the fitted material further comprises one selected from a group consisting of: a textile, a plastic, a polymer, a felt, a spunlace, a gauze, a nylon, a vinyl, a metal, and a combination thereof.

14. The biofeedback apparatus of claim 8, wherein the fitted material is sleeve, with at least one open end.

15. A biofeedback method, comprising:
providing a biofeedback apparatus, the biofeedback apparatus having:
a fitted material configured to a user's foot;
a plurality of sensing units operably associated with said fitted material;
a plurality of sensing unit transceivers operatively associated to each sensing unit of the plurality of sensing units, wherein said at least one sensing unit transceiver is operatively associated with said fitted material and said plurality of sensing units; and a data acquisition device, for acquiring at least one object data from said plurality of sensing unit transceivers;
fitting said biofeedback apparatus onto the user's foot;
locomoting said biofeedback apparatus with said user's foot;
sensing at least one variable with the plurality of sensing units;
communicating the at least one variable to a computer configured with computation
programming; and
analyzing the object data by the computer to determine a biofeedback of the foot.

16. The method of claim 15, further comprising preparing an incorporated biofeedback orthotic which incorporates said biofeedback.

17. The biofeedback method of claim 15, further wherein measuring comprises sensing at a plurality of locations with the biofeedback apparatus.

18. The biofeedback method of claim 15, wherein the at least one variable may be selected from the group consisting essentially of: pressure, displacement, strain, stress, static friction, kinetic friction, and combinations thereof.

19. The biofeedback method of claim 15, wherein the plurality of sensing units detects and/or records changes in pressure/tension of the body part.

20. The biofeedback method of claim 19 wherein the changes in pressure/tension of the body part is used in order to fit and orthotics.
